(19) United States
(12) Patent Application Publication
(10) Pub. No.: US 2009/0030500 A1
(43) Pub. Date: Jan. 29, 2009

(54) Iron Ion Releasing Endoprostheses

(76) Inventors: Jan Weber, Maastricht (NL); Peter Albrecht, Isen (DE)

Correspondence Address: FISH & RICHARDSON P.C., PO BOX 1022, MINNEAPOLIS, MN, 55440-1022, US

(21) Appl. No.: 11/829,585

(22) Filed: Jul. 27, 2007

(51) Int. Cl. A61F 2/06

(52) U.S. Cl. 623/1.15

(57) ABSTRACT

An endoprosthesis that includes a base portion and a source of Fe(II) ions that is compositionally distinct from the base portion and releasable from the endoprosthesis under physiological conditions.

### TECHNICAL FIELD

This invention relates to endoprostheses, and more particularly to stents.

### BACKGROUND

The body includes various passageways such as arteries, other blood vessels, and other body lumens. These passageways sometimes become occluded or weakened. For example, the passageways can be occluded by a tumor, restricted by plaque, or weakened by an aneurysm. When this occurs, the passageway can be reopened, reinforced, or even replaced with a medical endoprosthesis. An endoprosthesis is typically a tubular member that is placed in a lumen in the body. Examples of endoprostheses include stents, covered stents, and stent-grafts.

Endoprostheses can be delivered inside the body by a catheter that supports the endoprosthesis in a compacted or reduced-size form as the endoprosthesis is transported to a desired site. Upon reaching the site, the endoprosthesis is expanded, for example, so that it can contact the walls of the lumen.

The expansion mechanism can include forcing the endoprosthesis to expand radially. For example, the expansion mechanism can include a catheter carrying a balloon, which carries a balloon-expandable endoprosthesis. The balloon can be inflated to deform and to fix the expanded endoprosthesis at a predetermined position in contact with the lumen wall. The balloon can then be deflated, and the catheter withdrawn.

In another delivery technique, the endoprosthesis is formed of an elastic material that can be reversibly compacted and expanded, e.g., elastically or through a material phase transition. During introduction into the body, the endoprosthesis is restrained in a compacted condition. Upon reaching the desired implantation site, the restraint is removed, for example, by retracting a restraining device such as an outer sheath, enabling the endoprosthesis to self-expand by its own internal elastic restoring force.

Restenosis after endoprosthesis implantation can pose a serious problem. Migrating and proliferating smooth muscle cells (SMCs) responding to an initial injury accompanied by the deposition of the extracellular matrix are thought to be key events in causing restenosis.

### SUMMARY

An endoprosthesis is disclosed that includes a base portion and a source of Fe(II) ions that is compositionally distinct from the base portion and releasable from the endoprosthesis under physiological conditions.

In some embodiments, the source of Fe(II) ions can be implanted within the base portion. For example, the source of Fe(II) ions can be in the form of nano-particles implanted within the base portion. In some embodiments, the base portion can include pores and the source of Fe(II) ions can reside within the pores. In some embodiments, the source of Fe(II) ions can be in the form of a layer overlying the base portion. In some embodiments, the source of Fe(II) ions can be in the form of a wire. In some embodiments, the endoprosthesis can further include a drug eluting coating overlying the base portion. The drug eluting coating can include the source of Fe(II) ions. In some embodiments, the endoprosthesis can include a concentration gradient of Fe(II) ions.

In some embodiments, the source of Fe(II) ions can include metallic iron or an alloy thereof. For example, the source of Fe(II) ions an include iron that is at least 99% pure. The source of Fe(II) ions can also include iron alloyed with Mn, Ca, Si, or a combination thereof. In some embodiments, the source of Fe(II) ions can be iron oxides, iron carbides, iron sulfides, iron borides, or combinations thereof. For example, the source of Fe(II) ions can include magnetite.

In some embodiments, the base portion can include a metal alloy. For example, the metal alloy could be stainless steel, platinum enhanced stainless steel, cobalt-chromium alloys, nickel-titanium alloys, or a combination thereof.

In some embodiments, the base portion can include a bioerodable material, such as a bioerodable metal (e.g., magnesium or iron) or a bioerodable polymer. Examples of bioerodable polymers include polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly-L-lactide, poly-D-lactide, polyglycolide, and poly(alpha-hydroxy acid).

In some embodiments, the endoprosthesis can further include a porous coating overlying the base portion, the source of Fe(II) ions, or a combination thereof. For example, the porous coating can be a calcium phosphate hydroxy apatite coating, a sputtered titanium coating, a porous inorganic carbon coating, or a combination thereof.

In some embodiments, the endoprosthesis can be a stent.

A method of forming an endoprosthesis is also described. The method includes implanting Fe(II) ions into a surface of an endoprosthesis, such that the resulting endoprosthesis is adapted to release Fe(II) ions under physiological conditions. For example, the Fe(II) ions can be implanted using a metal ion immersion implantation process.

The details of one or more embodiments are set forth in the accompanying drawings and the description below. Other features, objects, and advantages will be apparent from the description and drawings, and from the claims.

### DESCRIPTION OF DRAWINGS

FIG. 1 is a perspective view of an example of an expanded stent.

FIG. 2 is a perspective view of an example of an expanded stent having an interwoven iron wire.

Like reference symbols in the various drawings indicate like elements.

### DETAILED DESCRIPTION

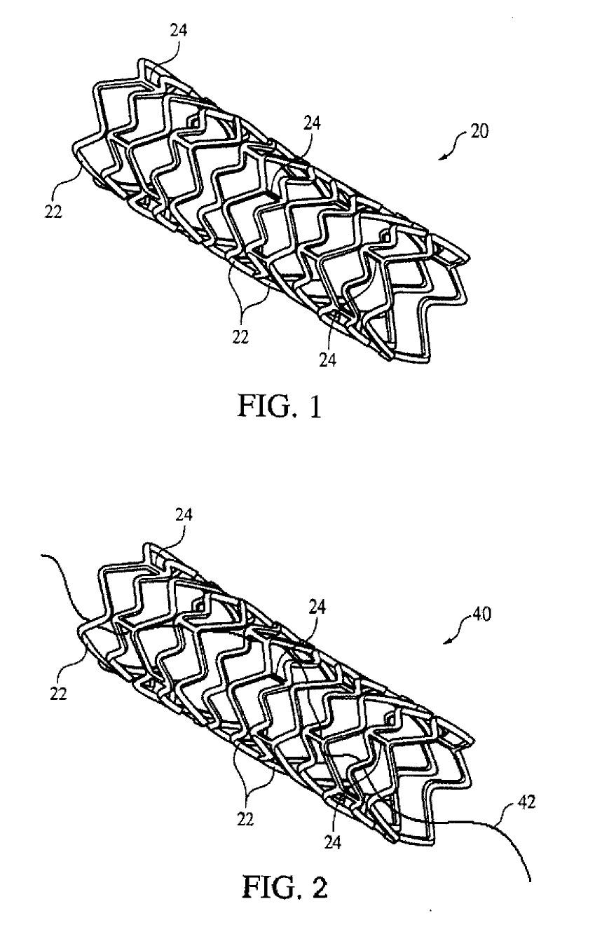

Referring to FIG. 1, a stent 20 can have the form of a tubular member defined by a plurality of bands 22 and a plurality of connectors 24 that extend between and connect adjacent bands. For example, the stent 20 in FIG. 1 can be a balloon-expandable stent. During use, bands 22 can be expanded from an initial, small diameter to a larger diameter to contact stent 20 against a wall of a vessel, thereby maintaining the patency of the vessel. Connectors 24 can provide stent 20 with flexibility and conformability that allow the stent to adapt to the contours of the vessel.

The stent 20 can include a base portion and a source of Fe(II) ions compositionally distinct from a base portion. The source of Fe(II) ions can be releasable from the stent 20 under physiological conditions. The resulting Fe(II) ions can inhibit at least some of the processes associated with cell proliferation. Accordingly, by providing a source of Fe(II) ions that can be released from a stent 20 under physiological conditions, the resulting Fe(II) ions released into a patient's body can inhibit smooth muscle cell proliferation, and thereby reduce the likelihood of restenosis.

The source of Fe(II) ions can take a variety of forms. For example, the source of Fe(II) ions can be Fe(II) ions implanted into portions of the stent 20. As will be described below, one possible method for implanting Fe(II) ions into portions of a stent 20 is by a metal ion immersion implantation process (MPIII).

The source of Fe(II) ions can also be in the form of a metallic iron or an alloy thereof. For example, iron can be alloyed with Mn, Ca and/or Si, which are all biocompatible. Some suitable iron alloys are described in, for example, Ototani U.S. Pat. No. 2,950,187. In some embodiments, the source of Fe(II) ions can be iron that is at least 99% pure. Metallic iron or alloys thereof can be in the form of coatings overlying all or a selected portion of a stent, nanoparticles implanted into all or a selected portion of a stent, or even a wire positioned between the stent and the vessel. Iron nanoparticles of very high purity (e.g., 99.999% by weight iron) are commercially available from American Elements, 1093 Broxton Ave. Suit 200, Los Angeles, Calif. 90024. High purity iron wire can be purchased from Goodfellow under the designation FE005105—Iron WireDiameter: 0.025 mm, High Purity: 99.99+% Temper.

The source of Fe(II) ions can be in the form of a bioerodable iron-containing ceramic or an iron salt. Examples include iron oxides, iron carbides, iron sulfides, iron borides, or a combination thereof. In some embodiments, the source of Fe(II) ions can be in the form of magnetite (Fe₃O₄). As magnetite degrades, it provides two Fe(III) ions for every Fe(II) ion, and therefore can provide a controlled release of Fe(II) ions. Magnetite can be in the form of nano- or micro-sized particles.

The base portion of a stent can be either a bioerodable or non-bioerodable material. Bioerodable base portions can be bioerodable metals and/or bioerodable polymers. For example, the base portion can include magnesium or an alloy thereof. The base portion can also be a pure iron, for example iron that is at least 99% pure. A bioerodable polymer base portion can include, for example, polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly-L-factidc, poly-D-lactide, polyglycolide, poly(alpha-hydroxy acid), or a combination thereof. In some embodiments, a bioerodable base portion can be substantially free of iron. A non-bioerodable base portion can include, for example, metal alloys such as stainless steel, platinum enhanced stainless steel, cobalt-chromium alloys, nickel-titanium alloys, or combinations thereof.

The source of Fe(II) ions can be in the form of a layer overlying the base portion. The source of Fe(II) ions can be a metallic iron or a biocrodable iron alloy. The base portion can be a bioerodable material or a non-biocrodable material. One method to produce an outer layer of iron on a base portion includes sputtering iron onto the base portion. Another possible method of producing a layer of iron on a base portion includes the use of pulsed laser deposition (PLD) or inverse PLD.

The source of Fe(II) ions can also be incorporated within a layer of another material overlying the base portion. For example, the source of Fe(II) ions can be in the form of nano-particles embedded within or Fe(II) ions implanted into a layer of biocrodable metal or biocrodable polymer. The source of Fe(II) ions can also reside within pores of a layer overlying the base portion. In some embodiments, the layer can be a drug eluting coating overlying a base portion. For example, the source of Fe(II) ions can be implanted into a conventional polymeric (e.g., SIBS) drug elution coating. In other embodiments, the source of Fe(II) ions can be in the form of nanoparticles implanted within the drug eluting coating or the drug eluting coating can include pores filled with a source of Fe(II) ions. Upon the release Fe(II) ions and the erosion of the source of Fe(II) ions out of the drug eluting coating, the drug-eluting coating can become more porous and thereby increase the drug release of the remaining drug molecules.

The source of Fe(II) ions can be in the form of Fe(II) ions implanted into the base portion. For example, the Fe(II) ions can be implanted by MPIII. The use of MPIII allows for the implantation of iron ions into complex 3D structures. The use of MPIII can result in an layer of implanted Fe(II) ions, a layer of metallic iron, or a combination thereof. The use of MPIII can also create a concentration gradient of Fe(II) ions in the base portion. In some embodiments, an MPIII treatment to implant Fe(II) ions can be followed by a second iron coating process.

In the case of a magnesium or magnesium alloy base portion, a layer of iron on top of the magnesium base portion can delay corrosion of the magnesium base portion when under physiological conditions. Accordingly, a magnesium-iron stent can be designed to not only inhibit smooth muscle cell proliferation but also to erode over a desired time period. For example, an outer layer of a magnesium stent could have up to 94% weight percent iron implanted within the magnesium or magnesium alloy. The use of MPIII can also result in a gradual transition of the iron into the magnesium, which can provide lower interfacial stress between the magnesium and iron layers.

A magnesium-iron strut could be formed by use of a layer-by-layer method. A magnesium base could be implanted with iron ions by use of MPIII and then additional layers of magnesium and iron applied by use of PLD and MPIII. This layer-by-layer approach can provide additional corrosion protection for the magnesium and supply Fe(II) ions throughout the life of the stent.

Fe(II) ions can also be implanted into bioerodable polymeric stents by an ion implantation process (e.g., by rotating the polymeric stent on top of a metallic holder), to result in a bioerodable polymeric base portion having implanted Fe(II) ions.

The source of Fe(II) ions can be the form of nano- or micro-particles embedded within the base portion. As discussed above, these nano- or micro-particles can include metallic iron or alloys thereof, iron containing ceramics, or iron salts (e.g., nano-particles of magnetite or of 99.999% pure iron).

Nano- or micro-particles can be incorporated into a base portion in a number of ways. For example, a stent can be formed by compounding nano- or micro-particles into a melt of biodegradable polymer. Another example includes adding particles in a variety of shells in the layer-by-layer method. The concentration of the nano- or micro-particles can vary from layer to layer. Nano-particles of a source of Fe(II) ions can also be embedded into a base portion by generating a stream of charged nanoparticles and placing a base portion into the stream by placing the base portion on an electrode and energized the electrode to have a polarity opposite to the charged particles. The stream of charged nanoparticles can be formed by forming a solution containing the nanoparticles, spraying the solution form a charged nozzle, and evaporating the solution. A more detailed description of a similar process for embedding nanoparticles into a polymeric medical device can be found in, for example, Weber, U.S. Pat. No. 6,803,070.

The base portion can include pores and the source of Fe(II) ions can reside within the pores. The base portion can be a non-bioerodable material or can also be a bioerodable material. By depositing the source of Fe(II) ions within pores of a base portion, the rate of corrosion of the Fe(II) ions can be controlled.

The stent can include a porous coating overlying the source of Fe(II) ions or overlying the base portion. The porous coating can be an inorganic coating, e.g., a calcium phosphate hydroxy apatite (CaHA) coating, a sputtered titanium coating, or a porous inorganic carbon coating. By providing a porous coating, direct contact between corroding iron and endothelial cells that cover the stent can be avoided.

FIG. 2 depicts an arrangement where the source of Fe(II) ions can be in the form of a wire 42. As shown, the wire 42 is interwoven with the body of the stent 40. At least a portion of the stent 40 forms a base portion. The wire can be positioned between the stent and the vessel to supply iron as the iron corrodes. This arrangement can provide a more uniform distribution of iron into the tissue. For example, a very thin wire that forms a higher dense network than the stent itself can be used. High purity iron wire can be purchased from Goodfellow under the designation FE005105—Iron WireDiameter: 0.025 mm, High Purity: 99.99+% Temper. The source of Fe(II) ions can also be an bioerodable iron alloy.

Stent 20 can be of a desired shape and size (e.g., coronary stents, aortic stents, peripheral vascular stents, gastrointestinal stents, urology stents, and neurology stents). Depending on the application, stent 20 can have a diameter of between, for example, 1 mm to 46 mm. In certain embodiments, a coronary stent can have an expanded diameter of from 2 mm to 6 mm. In some embodiments, a peripheral stent can have an expanded diameter of from 5 mm to 24 mm. In certain embodiments, a gastrointestinal and/or urology stent can have an expanded diameter of from 6 mm to about 30 mm. In some embodiments, a neurology stent can have an expanded diameter of from about 1 mm to about 12 mm. An abdominal aortic aneurysm (AAA) stent and a thoracic aortic aneurysm (TAA) stent can have a diameter from about 20 mm to about 46 mm. Stent 20 can be balloon-expandable, self-expandable, or a combination of both (e.g., U.S. Pat. No. 5,366,504).

In use, stent 20 can be used, e.g., delivered and expanded, using a catheter delivery system. Catheter systems are described in, for example, Wang U.S. Pat. No. 5,195,969, Hamlin U.S. Pat. No. 5,270,086, and Raeder-Devens, U.S. Pat. No. 6,726,712. Stents and stent delivery are also exemplified by the Sentinol® system, available from Boston Scientific Scimed, Maple Grove, Minn.

Stent 20 can be a part of a covered stent or a stent-graft. In other embodiments, stent 20 can include and/or be attached to a biocompatible, non-porous or semi-porous polymer matrix made of polytetrafluoroethylene (PTFE), expanded PTFE, polyethylene, urethane, or polypropylene.

The arrangements described herein can be used to form other endoprostheses, e.g., to form a guidewire or a hypotube.

All publications, references, applications, and patents referred to herein are incorporated by reference in their entirety.

Other embodiments are within the claims.

What is claimed is:

1. An endoprosthesis comprising a base portion and a source of Fe(II) ions that is compositionally distinct from the base portion and releasable from the endoprosthesis under physiological conditions.

2. The endoprosthesis of claim 1, wherein the source of Fe(II) ions is implanted within the base portion.

3. The endoprosthesis of claim 1, wherein the source of Fe(II) ions is in the form of nano-particles implanted within the base portion.

4. The endoprosthesis of claim 1, wherein the base portion comprises pores and the source of Fe(II) ions resides within the pores.

5. The endoprosthesis of claim 1, wherein the source of Fe(II) ions is in the form of a layer overlying the base portion.

6. The endoprosthesis of claim 1, wherein the source of Fe(II) ions is in the form of a wire.

7. The endoprosthesis of claim 1, further comprising a drug eluting coating overlying the base portion, wherein the drug eluting coating comprises the source of Fe(II) ions.

8. The endoprosthesis of claim 1, comprising a concentration gradient of Fe(II) ions in the endoprosthesis.

9. The endoprosthesis of claim 1, wherein the source of Fe(II) ions comprises metallic iron or an alloy thereof.

10. The endoprosthesis of claim 1, wherein the source of Fe(II) ions comprises iron that is at least 99% pure.

11. The endoprosthesis of claim 1, wherein the source of Fe(II) ions comprises iron alloyed with an element selected from the group consisting of Mn, Ca, Si, and combinations thereof.

12. The endoprosthesis of claim 1, wherein the source of Fe(II) ions is selected form the group consisting of iron oxides, iron carbides, iron sulfides, iron borides, and combinations thereof.

13. The endoprosthesis of claim 1, wherein the source of Fe(II) ions comprises magnetite.

14. The endoprosthesis of claim 1, wherein the base portion comprises a metal alloy selected from the group consisting of stainless steel, platinum enhanced stainless steel, cobalt-chromium alloys, nickel-titanium alloys, and combinations thereof.

15. The endoprosthesis of claim 1, wherein the base portion comprises a bioerodable material.

16. The endoprosthesis of claim 1, wherein the base portion comprises magnesium.

17. The endoprosthesis of claim 1, wherein the base portion comprises iron.

18. The endoprosthesis of claim 1, wherein the base portion comprises a bioerodable polymer selected from the group consisting of polydioxanone, polycaprolactone, polygluconate, polylactic acid-polyethylene oxide copolymers, modified cellulose, collagen, poly(hydroxybutyrate), polyanhydride, polyphosphoester, poly(amino acids), poly-L-lactide, poly-D-lactide, polyglycolide, poly(alpha-hydroxy acid), and combinations thereof.

19. The endoprosthesis of claim 1, further comprising a porous coating overlying the base portion, the source of Fe(II) ions, or a combination thereof.

20. The endoprosthesis of claim 19, wherein the porous coating is selected from the groups consisting of calcium phosphate hydroxy apatite coatings, sputtered titanium coatings, porous inorganic carbon coatings, and combinations thereof.

21. The endoprosthesis of claim 1, wherein the endoprosthesis is a stent.

22. An endoprosthesis comprising a base portion comprising magnesium or an alloy thereof and a source of Fe(II) ions that is distinct from the base portion and releasable from the endoprosthesis under physiological conditions, the source of Fe(II) ions comprising metallic iron or an alloy thereof.

23. The endoprosthesis of claim 22, comprising a concentration gradient of Fe(II) ions in the endoprosthesis.

24. A method of forming an endoprosthesis comprising
implanting Fe(II) ions into a surface of an endoprosthesis or a precursor thereof, wherein the resulting endoprosthesis is adapted to release the Fe(II) ions under physiological conditions.

25. The method of claim 24, wherein the Fe(II) ions are implanted using a metal ion immersion implantation process.
